(19) United States
(12) Patent Application Publication
(10) Pub. No.: US 2024/0277717 A1
(43) Pub. Date: Aug. 22, 2024

(54) APPLICATION OF GANCICLOVIR IN PREPAPRATION OF MEDICINE FOR TREATING ALLERGIC RHINITIS

(71) Applicant: Union Hospital, Tongji Medical College, Huazhong University of Science and Technology, Wuhan (CN)

(72) Inventor: Jianjun Chen, Wuhan (CN)

(21) Appl. No.: 18/358,958

(22) Filed: Jul. 26, 2023

Related U.S. Application Data
(63) Continuation of application No. PCT/CN2023/096854, filed on May 29, 2023.

(30) Foreign Application Priority Data
Feb. 16, 2023 (CN) 2023101293738

(51) Int. Cl. A61K 31/522; A61K 45/06; A61P 37/08

(52) U.S. Cl. CPC A61K 31/522; A61K 45/06; A61P 37/08

(57) ABSTRACT

Ganciclovir (GCV) is a heterocyclic compound containing a purine ring. An application of the GCV in preparation of a medicine for treating allergic rhinitis is provided, in which the GCV is used as an only active component or use the GCV in combination with another medicine. Studies have shown that the GCV can effectively ameliorate symptoms of AR (allergic rhinitis) patients who are ineffective to anti-allergic medicine treatment in clinical and improve their quality of life. It is found from transcriptomics that the GCV regulates the function of nasal mucosal cilium cells, goblet cells and leukocytes. It is also found that the GCV may change immune balance of body by increasing regulatory T cells, thereby alleviating allergic symptoms in vitro peripheral blood mononuclear cells (PBMCs) medicine test.

### CROSS-REFERENCE TO RELATED APPLICATION

The application claims priority to Chinese patent application No. 202310129373.8, filed on Feb. 16, 2023, and entitled with “APPLICATION OF GANCICLOVIR IN PREPAPRATION OF MEDICINE FOR TREATING ALLERGIC RHINITIS”, and the entire contents of the above-mentioned application are hereby incorporated herein by reference.

### TECHNICAL FIELD

The disclosure relates to heterocyclic compounds containing a purine ring, and more particularly to an application/use of ganciclovir in preparation of a medicine for treating allergic rhinitis.

### BACKGROUND

Allergic rhinitis (AR) is one of a most common allergic diseases, with a global incidence rate of 10-25%, which seriously affects the quality of life of the affected population and increases a social and economic burden. AR is characterized by nasal hyperresponsiveness symptoms, such as rhinocleisis, rhinocnesmus, sneezing and rhinorrhea, with a clinical histories ranging from months to years. Treatments for the AR include patient educations, allergen avoidance, anti-allergic medicines and allergen-specific immunotherapy (AIT). Common anti-allergic medicines include nasal glucocorticoid sprays, leukotriene receptor antagonists, nasal spray or oral antihistamines and so on. Most of AR patients will gradually alleviate their rhinitis symptoms after the treatment with the anti-allergic medicines. The AIT is mainly used in the patients with moderate to severe AR, but the patients with the moderate to severe AR can also alleviate their nasal symptoms through routine anti-allergic medicines. However, there are some patients in clinical whose AR symptoms are not controlled even after receiving the best and maximum anti-allergic medicines, and, which is called intractable AR. Poor control of the AR symptoms may be related to some reasons such as inappropriate treatment options, poor patient compliance, social psychological factors, and therapy resistance. In addition to the first three reasons, although some patients alternately use different brands of the routine anti-allergic medicines, the nasal symptoms are still not controlled. The reasons for poor therapeutic responses of the some patients to the routine anti-allergic medicines are still unclear.

Ganciclovir (GCV) is an effective anti-viral medicine for treating deoxyribonucleic acid (DNA) viruses, such as cytomegalovirus and herpe svirus infections. A main mechanism of the GCV is that the GCV is phosphorylated by thymidine kinase of virus and then the GCV enters cells, inhibiting DNA polymerases and thereby inhibiting viral replication. From the known medicine mechanism, the GCV has no effect on allergic inflammation.

The applicant accidentally discovered some patients using GCV to treat other diseases in clinical, nasal allergic symptoms of the patients (without signs associated with viral infections) are significantly alleviated. After a period of treatment with the GCV, the anti-allergic medicines that originally have poor efficacy can regain the efficacy, effectively and lastingly ameliorating the nasal symptoms in the patients with the intractable AR. After a long-term careful observation, it is found that more than a half of the patients with the intractable AR receive significant alleviation in the AR symptoms after using the GCV.

### SUMMARY

Technical problems to be solved in the disclosure are aimed at the above shortcomings in related art, the disclosure provides an application of ganciclovir (GCV) in preparation of a medicine for treating allergic rhinitis (AR), which has a good clinical effect on the treatment of the AR, especially for intractable AR that using the routine treatment methods is ineffective, and can significantly ameliorate clinical symptoms of patients.

To solve the above technical problems, technical solutions provided in the disclosure are as follow.

Specifically, an application of GCV in preparation of a medicine for treating AR is provided.

In an embodiment, clinical symptoms of the AR are at least one selected from the group consisting of rhinocleisis, rhinocnesmus, sneezing and rhinorrhea.

In an embodiment, the GCV is used as an only active component or the GCV in combination with another medicine ameliorating the AR.

In an embodiment, using the GCV in combination with the another medicine ameliorating the AR includes using the GCV first, then using the another medicine ameliorating the AR as needed or using the GCV in combination with the another medicine ameliorating the AR simultaneously.

In an embodiment, the another medicine ameliorating the AR include at least one of a nasal spray hormone and an oral antihistamine.

In an embodiment, the nasal spray hormone includes one of mometasone furoate, budesonide, triamcinolone acetonide and fluticasone; and the oral antihistamine includes one of loratadine, cetirizine and ebastine.

In an embodiment, the using the ganciclovir first, then using the other medicines ameliorating the allergic rhinitis as needed includes: using the GCV for 7-14 days first, then continually using the other medicines ameliorating the allergic rhinitis for 7-14 days as needed.

In an embodiment, forms of the prepared medicine using the ganciclovir include: capsules, granules, tablets, pulvis, ointments, powders, and aqueous solutions prepared by mixing pharmaceutically acceptable excipients with one of the ganciclovir as the only active component and a combination of the ganciclovir and other oral medicines ameliorating the allergic rhinitis.

In an embodiment, a dosage of the GCV is in a range of 250-500 milligrams per time, twice a day for 14 years old and above by oral administration, and 125-250 milligrams per time, twice a day for under 14 years old by oral administration.

Anti-allergic treatments for a class of AR patients in clinic are completely ineffective and the class of the AR patients has no history of viral infections, while GCV treatment can effectively ameliorate nasal symptoms of the class of the AR patients. During experiments, using the GCV to stimulate peripheral blood mononuclear cells (PBMCs) in vitro, a proportion of regulatory T cells (cluster of differentiation 4⁺ Forkhead box protein P3⁺ regulatory T cells, abbreviated as CD4⁺FOXP3⁺T cells) is detected by a flow cytometry, finding that the regulatory T cells in an effective group of the GCV treatment can be significantly increased after stimulation, and the mechanism of the CGV may be that the GCV changes body's immune balance by increasing the regulatory T cells to thereby alleviate the allergic symptoms.

The beneficial effect of the disclosure is as follows. In the disclosure, the application of the GCV in the preparation of the medicine for treating the AR is provided. Research has shown that the GCV can effectively ameliorate the symptoms and improve qualities of life of the AR patients who are ineffective to anti-allergic medicines treatment in clinical. It is also found from transcriptomics that the GCV regulates the function of nasal mucociliary cells, goblet cells and leucocytes. The medicine test of PBMCs in vitro finds that the GCV may change the body's immune balance by increasing the regulatory T cells to thereby alleviate the allergic symptoms.

### BRIEF DESCRIPTION OF DRAWINGS

In order to illustrate technical solutions in embodiments of the disclosure more clearly, a brief introduction will be given to the accompanying drawings required in the description of the embodiments. Apparently, the accompanying drawings in the following description are some embodiments of the disclosure. For those skilled in the art, other accompanying drawings can be obtained based on these drawings without any creative effort.

FIGS. 1A-1C illustrate score comparison diagrams of allergic rhinitis control test (ARCT), visual analogue scale (VAS) and total nasal symptom score (TNSS) of patients (n=26) in a ganciclovir (GCV) effective group in diagnosis and treatment process according to embodiment 1 of the disclosure.

FIG. 2 illustrates a comparison diagram of nasal symptom scores before and after GCV treatment for the patients in the GCV effective group according to the embodiment 1 of the disclosure.

FIG. 3A illustrates a comparison diagram of gene abundance of Epstein-Barr virus (EB virus), human-cytomegalovirus, human-herpes virus (HSV) 1, 2, 3, 6, 7, 8 in nasal scraping cells before treatment between a common allergic rhinitis (AR) group and the GCV effective group.

FIG. 3B illustrates a comparison diagram of gene abundance of EB virus, human-cytomegalovirus, HSV 1, 2, 3, 6, 7, 8 before and after treatment in the GCV effective group according to the embodiment 1 of the disclosure.

FIG. 4 illustrates a volcano plot of ribonucleic acid sequencing (RNA-seq) differential genes of the nasal scraping cells before treatment in the GCV effective group (n=6) and the common AR group (n=5) according to the embodiment 1 of the disclosure.

FIG. 5 illustrates a pathway of up-regulated differential gene enrichment in the GCV effective group compared with the common AR group according to the embodiment 1 of the disclosure.

FIG. 6 illustrates a pathway of down-regulated differential gene enrichment in the GCV effective group compared with the common AR group according to the embodiment 1 of the disclosure.

FIG. 7 illustrates a volcano plot of the RNA-seq differential genes of the nasal scraping cells before treatment in the GCV effective group (n=6) and a GCV ineffective group (n=3) according to the embodiment 1 of the disclosure.

FIG. 8 illustrates a pathway of up-regulated differential gene enrichment in the GCV effective group compared with the GCV ineffective group according to the embodiment 1 of the disclosure.

FIG. 9 illustrates a pathway of down-regulated differential gene enrichment in the GCV effective group compared with the GCV ineffective group according to the embodiment 1 of the disclosure.

FIG. 10 illustrates a cluster heat map of RNA-seq differential genes of the nasal scraping cells before and after treatment in the GCV effective group (n=6) according to the embodiment 1 of the disclosure.

FIG. 11 illustrates a pathway of up-regulated differential gene enrichment in the GCV effective group after treatment compared with that before treatment according to the embodiment 1 of the disclosure.

FIG. 12 illustrates a pathway of down-regulated differential gene enrichment in the GCV effective group after treatment compared with that before treatment according to the embodiment 1 of the disclosure.

FIG. 13 illustrates a flow cytometry diagram of cluster of differentiation 4⁺ (CD4⁺) Forkhead box protein P3⁺ (FOXP3⁺) cells in peripheral blood mononuclear cells (PBMCs) stimulated by GCV in vitro in the GCV effective group and the GCV ineffective group according to the embodiment 1 of the disclosure.

FIGS. 14A-14B illustrate comparison diagrams of percentage of CD4⁺ FOXP3⁺ cells/CD4⁺ cells in the PBMCs stimulated by GCV in vitro between the GCV effective group and the GCV ineffective group according to the embodiment 1 of the disclosure.

FIG. 15 illustrates a flow cytometry diagram of CD4⁺ interferon gamma+(IFNγ+) cells in the PBMCs stimulated by GCV in vitro in the GCV effective group and the GCV ineffective group according to the embodiment 1 of the disclosure.

FIGS. 16A-16B illustrate comparison diagrams of percentage of CD4⁺ IFNγ+ cells/CD4⁺ cells in the PBMCs stimulated by GCV in vitro between the GCV effective group and the GCV ineffective group according to the embodiment 1 of the disclosure.

FIG. 17 illustrates a flow cytometry diagram of CD4⁺ interleukin 4⁺ (IL4⁺) cells in the PBMCs stimulated by GCV in vitro in the GCV effective group and the GCV ineffective group according to the embodiment 1 of the disclosure.

FIG. 18A-18B illustrate comparison diagrams of percentage of CD4⁺ IFNγ+ cells/CD4⁺ cells in the PBMCs stimulated by GCV in vitro between the GCV effective group and the GCV ineffective group according to the embodiment 1 of the disclosure.

FIG. 19 illustrates a flow cytometry diagram of CD4⁺ interleukin-17A+(IL17A⁺) cells in the PBMCs stimulated by GCV in vitro in the GCV effective group and the GCV ineffective group according to the embodiment 1 of the disclosure.

FIGS. 20A-20B illustrate comparison diagrams of percentage of CD4⁺ IL17A⁺ cells/CD4⁺ cells in the PBMCs stimulated by GCV in vitro between the GCV effective group and the GCV ineffective group according to the embodiment 1 of the disclosure.

FIG. 21 illustrates a flow cytometry diagram of CD8⁺ IFNγ⁺ cells in the PBMCs stimulated by GCV in vitro in the GCV effective group and the GCV ineffective group according to the embodiment 1 of the disclosure.

FIGS. 22A-22B illustrate comparison diagrams of percentage of CD8⁺ IFNγ⁺ cells/CD4⁺ cells in the PBMCs stimulated by GCV in vitro between the GCV effective group and the GCV ineffective group according to the embodiment 1 of the disclosure.

FIG. 23 illustrates a scatter diagram of changes in TNSS and VAS before and after treatment with GCV and changes in proportion of Foxp3⁺ Treg cells stimulated by the GCV according to the embodiment 1 of the disclosure.

FIG. 24 illustrates a receiver operating characteristic (ROC) curve of changes in proportion of Foxp3⁺ Treg cells in the GCV effective group and the GCV ineffective group according to the embodiment 1 of the disclosure.

FIG. 25 illustrates a flowchart of specific diagnosis and treatment process, where a standard of “GCV treatments are effective^{#}” is ARCT score≥20 points, and a standard of “GCV treatments are ineffective^{#}” is ARCT score<20 points.

### DETAILED DESCRIPTION OF EMBODIMENTS

In order to enable those skilled in the art to understand technical solutions of the disclosure better, the following is a further detailed description of the disclosure in conjunction with the accompanying drawings.

### Embodiment 1

### Study of Ganciclovir (GCV) for Intractable Allergic Rhinitis (AR)

### 1.1 Research Objects

All patients are recruited from outpatient department of otolaryngology, the patients with AR complain of poor efficacy of routine anti-allergic medicines (nasal glucocorticoid sprays, oral antihistamines, nasal antihistamine sprays and oral leukotriene receptor antagonists). The inclusion and exclusion criteria of recruiting are as follows.

The inclusion criteria: (1) patients have two or more typical nasal hyperresponsiveness symptoms (rhinocleisis, rhinocnesmus, sneezing and rhinorrhea), at least one positive allergen from a skin prick test or a serologically allergen-specific immunoglobulin E (IgE) test; (2) the patients' courses of disease last at least 6 months; (3) the patients' severities of nasal symptoms are one of moderate symptom and severe symptom; (4) cytological smears of nasal secretions show that a proportion of eosinophile granulocytes in total inflammatory cells is more than 10%; (5) the patients complain that the routine anti-allergic medicines are ineffective.

The exclusion criteria: (1) patients have viral/bacterial infection symptoms, fevers and/or other systemic manifestations in the past 2 weeks; (2) patients have other nasal diseases, such as nasal polyps or nasal tumors; (3) patients have a history of nasal surgery in the past 6 months; (4) patients are allergic to the GCV; (5) patients are pregnant women and patients are preparing for pregnancy; (6) the patients have severe neutropenia (absolute neutrophil count<0.5×10⁹ per liter) or severe thrombocytopenia (platelet count<2.5×10¹⁰ per liter).

An interventional non-randomized cohort study is conducted. Selected patients are treated with the routine anti-allergic medicines for 2 weeks to exclude the illusion of poor efficacy due to non-standard medication (combined use of nasal spray hormones and oral antihistamines to exclude treatment, and the selected patients are switched to with another brand of similar medicines if the selected patients have previously used the above medicines but no effect).

The selected patients are grouped according to responses to the routine anti-allergic medicines. If the routine anti-allergic medicines treatments are effective, the selected patients are classified as a common AR group; if ineffective, the selected patients are defined as intractable AR patients. The intractable AR patients are treated with the GCV for 2 weeks (oral, 500 milligrams per time (mg/time) twice a day for the patients≥14 years old; 250 mg/time twice a day for the patients<14 years old). If the GCV treatments are ineffective, the patients are classified as a GCV ineffective group; if effective, the patients are classified as GCV effective group. After 2 weeks of the GCV treatment, the patients in the GCV effective group, if total nasal symptom score (TNSS)≥4 or visual analogue scale (VAS)≥3 centimeters (cm) after the GCV treatment, the patients continue to use the routine anti-allergic medicines for 2 weeks after the GCV treatment, conversely, the GCV treatment ends. The nasal symptoms are evaluated at each follow-up. In this situation, nasal secretion smears, nasal secretions, nasal scraping epithelial cells and peripheral blood are collected.

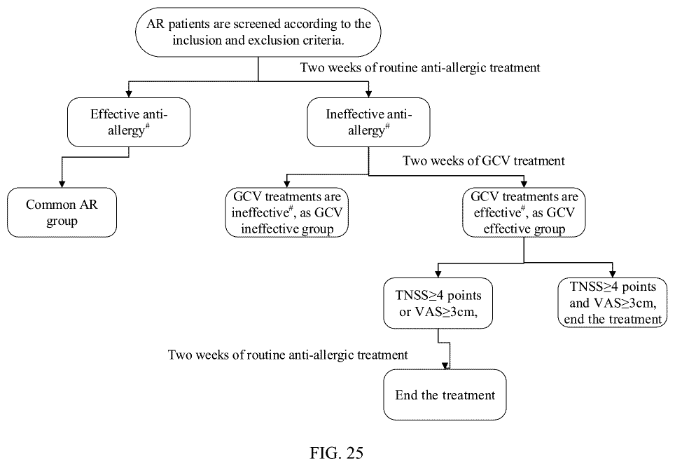

### Specific Diagnosis and Treatment Process is Shown in FIG. 25.

### 1.2 Clinical Symptoms Score

The visual analogue scale (VAS): the severity of all comprehensive symptoms is assessed by the VAS, ranging from 0 (no symptoms) to 10 centimeters (most severe and imaginable symptoms). Allergic rhinitis and its impact on asthma (ARIA) 2008 guideline classification: “mild” AR=0-3 centimeters, “moderate” AR=3.1-7 centimeters, “severe” AR=7.1-10 centimeters.

Allergic rhinitis control test (ARCT) is a self-administered questionnaire on AR control verified by Demoly et al. It consists of five questions with a score of 1-5 points for each question scored and a total score of 5-25 points. The intractable AR is defined as patients with ARCT score<20 points after 2 weeks of treatment.

Nasal symptom score: rhinocleisis, rhinocnesmus, sneezing, rhinorrhea are scored separately. No symptoms=0; mild (mild symptoms, easy to tolerate)=1; moderate (obvious symptoms, annoying, but tolerable)=2; severe (symptoms are not tolerable, affecting daily life or sleep)=3. The total nasal symptom score (TNSS) is the sum of all nasal symptom scores, ranging from 0 to 12.

### 1.3 Nasal Secretion Smears

The patients blow their nose (both sides), or gently rotate a glass rod at a front end of the patients' nasal vestibule to obtain the nasal secretions, avoiding rubbing the mucosal surface as much as possible. The obtained nasal secretions are evenly and lightly coated on slides, and a coverage area of the nasal secretion smear is required to exceed ⅔ of the slides. After natural air drying, the slides are dyed with Wright-Giemsa stain (Baso Diagnostics Inc. Zhuhai; China). After dyeing, cells are counted under an optical microscope. A percentage of eosinophils in inflammatory cells under 10×100 times microscope is observed, and an average value of 5 visual fields is taken as a result.

### 1.4 Collection and Detection of the Nasal Secretions

Two pieces of 1.0 centimeter (cm)×1.5 cm sterile medical sponge are gently placed into the patient's bilateral nasal cavity for 1-5 minutes (min). After the sponge adsorbs enough the nasal secretions, the sponge is gently removed and put into a 2 milliliters (mL) syringe. The nasal secretions are squeezed out and collected in a 1.5 mL centrifuge tube, centrifuged at 3000 revolutions per minute (rpm) for 5 min, and the supernatant is taken and placed in a refrigerator at −80° C. for testing. Eosinophil cationic protein (ECP) in the nasal secretions is detected by enzyme-linked immunosorbent assay (ELISA). The ECP-ELISA kit is from Zhongmin Taike Biotechnology Co., Ltd. Cytokine levels of interleukin (IL)-2, IL-4, IL-5, IL-6, IL-13, IL-17A, IL-28A, IL-33, interferon (IFN)-α, IFN-β, IFN-γ, IFN-λ, and Eotaxin in the nasal secretions are measured by using Luminex (R&D; LXSAHM-13).

### 1.5 Transcriptome Sequencing of Nasal Scraping Cells

After cleaning the nasal cavity, a mucosal epithelium of bilateral inferior turbinate is gently scraped with a medical sterile scraper, about a size of mung bean. The scraped mucosal epithelial cells are placed in a TRIzol™ reagent (Invitrogen™) and stored in liquid nitrogen for processing. Library preparation and high-throughput sequencing, data analysis, ribonucleic acid (RNA) sequence and deoxyribonucleic acid (DNA) virus sequence enrichment analysis are performed by Seqhealth Technology Co., Ltd. (China; wuhan). Sample quality analysis is performed by Nanodrop™ OneC spectrophotometer to detect expression at A260 and A280. Qualified samples are finally quantified by QubitR®3.0 fluorometer and Qubit™ RNA broad-range (BR) Assay kits. The original data is first filtered by Trimomatic (Version 0.36). Counting by FeatureCounts, and then calculate reads per kilobase of exon model per million mapped reads (RPKM). Differentially expressed genes between groups are identified using an edgeR package (Version 3.12.1). In analysis, P value<0.05 and fold change (FC) higher than 2 are considered to be significant differences in gene expression between groups.

1.7 GCV Medicine Test of the PBMCs in vitro.

The peripheral blood of patients with the intractable AR is collected. The peripheral blood mononuclear cells (PBMCs) are isolated within 8 hours using a lymphocyte separation medium (MP biomedicals) and suspended in Roswell Park Memorial Institute (RPMI) 1640 medium (Gibco™) containing 10% heat-inactivated fetal bovine serum (Gibco™, Grand Island) and 1% penicillin and streptomycin (Gibco™) at a density of 1×10⁶/mL. Three wells are set for each sample, including: a non treated well (also referred to as NT well), a well adding 1 microliter (μL) dimethyl sulfoxide (DMSO; Solarbio®) (also referred to as DMSO well), and a well adding 400 micromoles per liter (μM) of the GCV (diluted with DMSO, Selleck) (also referred to as GCV well). The PBMCs are cultured in a 24-well cell culture plate (Corning®, NY) under different stimulations in a 5% CO₂, 37° C. cell incubator. According to the instructions of Leukocyte Activation Cocktail with BD GolgiPlug™ (BD Bioscience), the PBMCs are added to a cell culture medium 4-6 hours before dyeing to improve the dyeing effect of intracellular cytokines. After 48 hours of cell culture, the cell solution is collected and dyed with anti-human fixable viability stain 510 (FVS-BV510), brilliant violet 605 anti-human cluster of differentiation 4 antibody (CD4-BV605), and cluster of differentiation 4-fluorescein isothiocyanate isomer I (CD8-FITC), and then dyed with interleukin-4-brilliant violet™ 421 dyes (IL-4-BV421), interleukin-17A-phycoerythrin (IL-17A-PE), human interferon-gamma-PE-cyanine7 dye (IFNγ-PE-Cy7), and FOXP3-BB700 (all from BD Bioscience) after breaking the nucleus with a fixed nuclear breaker (eBioscience™). BD LSRFortessa™ X-20 (BD Biosciences, USA) and FlowJo™ software (FlowJo, Ashland, OR USA) are used for analysis.

### 1.8 Statistical Analysis

The statistical analysis is performed using Graphpad Prism9.0 software, and data of cell subsets are analyzed using the FlowJo™ software (version 10.8.1, FlowJo.LLC, Ashland, OR USA), with basic information expressed as mean±standard deviation. If the data conform to a normal distribution, paired/unpaired t-test and common one-way analysis of variance (ANOVA) are performed. If the data do not conform to the normal distribution, the nonparametric test is used. P<0.05 is considered statistically significant difference, * means P<0.05, ** means P<0.01, *** means P<0.001, **** means P<0.0001.

### 1.9 Medical Ethics

The study is approved by the Ethics Committee of Union Hospital, Tongji Medical College, Huazhong University of Science and Technology ([2022] 0221-01).

### 2. Experimentation Results

2.1 According to the inclusion criteria and the exclusion criteria, after a corresponding treatment observation, all the selected patients can be divided into the following three groups, the basic information is shown in Table 1.

TABLE 1
Basic information of patients

| | | | GCV | GCV | |
|---|---|---|---|---|---|
| | | Common | effective | ineffective | |
| | | AR group | group | group | P |
| Case number (male/female) | | 23 (9/14) | 26 (11/13) | 14 (7/7) | 0.18 |
| Age (year) | | 37.57 ± 16.48 | 29.04 ± 13.05 | 32.08 ± 10.59 | 0.11 |
| Course of disease (year) | | 5.35 ± 5.91 | 6.05 ± 5.92 | 7.32 ± 6.62 | 0.63 |
| VAS | | 8.41 ± 1.35 | 8.48 ± 1.33 | 8.63 ± 2.06 | 0.91 |
| Total nasal symptom score | rhinocleisis | 2.41 ± 0.94 | 2.50 ± 0.64 | 2.14 ± 0.91 | 0.42 |
| | rhinocnesmus | 1.86 ± 1.06 | 1.85 ± 1.03 | 1.79 ± 1.15 | 0.98 |
| | rhinorrhea | 2.50 ± 0.72 | 2.15 ± 0.99 | 2.21 ± 0.86 | 0.35 |
| | sneezing | 2.18 ± 0.78 | 1.92 ± 0.92 | 2.07 ± 0.80 | 0.56 |
| | TNSS | 8.57 ± 2.73 | 8.42 ± 2.12 | 8.21 ± 2.54 | 0.91 |
| ARCT | | 5.70 ± 1.85 | 5.77 ± 1.56 | 6.57 ± 3.16 | 0.43 |
| Accompanied by asthma (case) | | 1 | 4 | 0 | — |
| Total serum IgE (kU/L) | | 193.88 ± 302.41 | 360.76 ± 438.09 | 284.17 ± 260.81 | 0.64 |
| Serum SAA^{‡} (ng/mL) | | 658.70 ± 144.86 | 546.91 ± 131.38 | 546.97 ± 114.66 | 0.17 |
| Serum CRP^{§} (pg/mL) | | 767.0 ± 188.76 | 840.93 ± 203.16 | 723.47 ± 218.47 | 0.47 |
| Peripheral blood wbc (G/L) | | 7.58 ± 1.80 | 5.95 ± 1.73 | 6.20 ± 1.32 | |
| Peripheral blood eosinophils (G/L) | | 0.57 ± 0.19 | 0.36 ± 0.17 | 0.51 ± 0.6 | 0.17 |
| Peripheral blood lymphocyte (G/L) | | 2.19 ± 0.39 | 1.88 ± 0.63 | 1.94 ± 0.35 | 0.69 |
| Peripheral blood mononuclear cells (G/L) | | 0.45 ± 0.10 | 0.45 ± 0.17 | 0.44 ± 0.05 | 0.97 |
| Nasal secretion eosinophils/inflammatory | | 83.33 ± 26.67 | 82.25 ± 28.84 | 93.33 ± 9.43 | 0.71 |
| cells (%) | | | | | |

In Table 1, SAA^{‡} denotes serum amyloid A, and CRP^{§} denotes C-reactive protein.
2.2 The GCV significantly ameliorates the nasal symptoms in the patients with the intractable AR

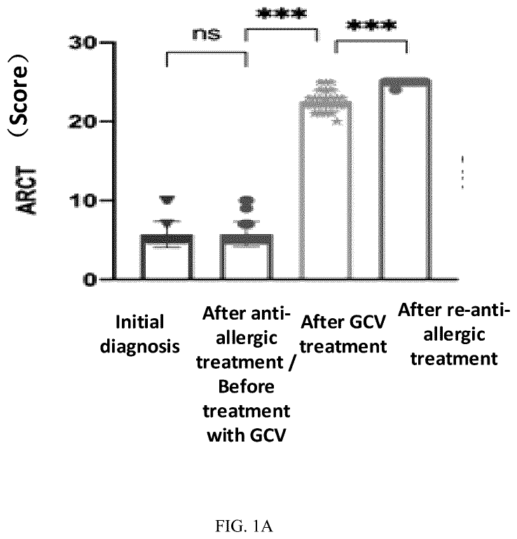

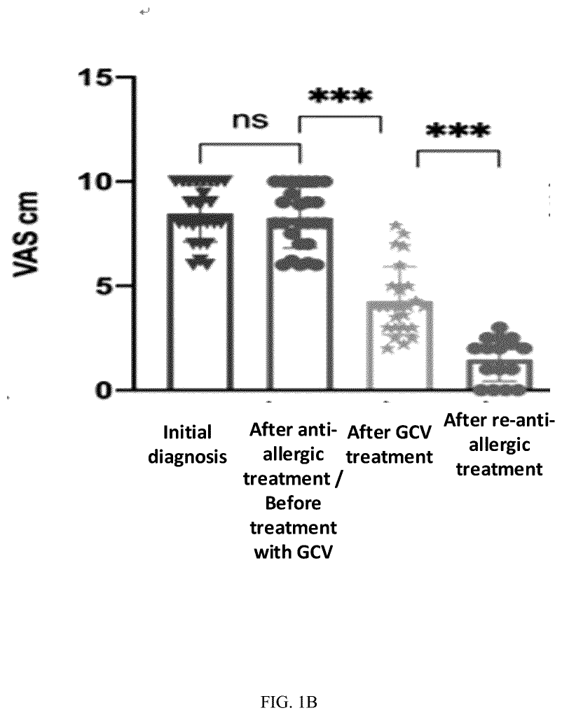

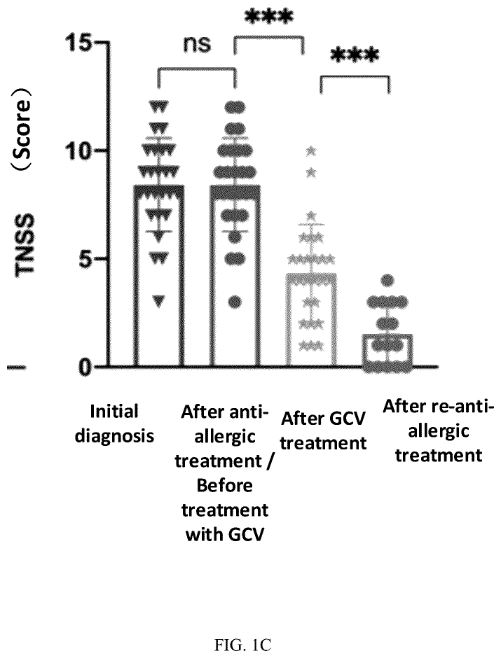

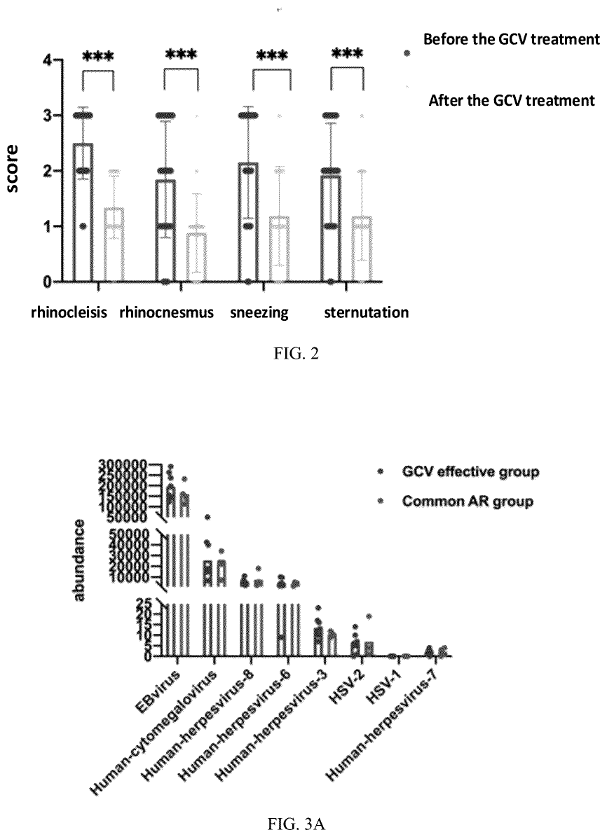

After 2 weeks of routine anti-allergic medicine treatment, according to the ARCT score, 63.5% (40/63) of the intractable AR patients receive the GCV treatment after the failure of the routine anti-allergic medicine treatment, among the intractable AR patients, 35% (14/40) are ineffective after the GCV treatment, and 65% (26/40) have significant improvement in VAS, TNSS and ARCT scores after the GCV treatment. The statistical analysis of the clinical symptom scores of the selected patients in the GCV effective group after the initial diagnosis, 2 weeks of the routine anti-allergic medicine treatment, the GCV treatment, and re-routine anti-allergic medicine treatment. Results are shown in FIGS. 1A-1C, FIGS. 1A-1C are comparison diagrams of the ARCT, VAS and TNSS scores of the patients in the GCV effective group (n=26) during the diagnosis and treatment. NS indicates p>0.05, * indicates p<0.05, ** indicates p<0.01, *** indicates p<0.001. It can be seen that the GCV significantly ameliorates the nasal symptoms of the AR patients who are ineffective with the routine anti-allergic medicines, after two weeks of the GCV treatment, a part of patients (n=15) with TNSS≥4 or VAS≥3 cm are treated with previously ineffective anti-allergic medicines for 2 weeks, which shows that the previously ineffective anti-allergic medicines could further reduce the nasal symptoms of the ineffective group patients. FIG. 2 is a comparison diagram of the nasal symptoms (rhinocleisis, rhinocnesmus, sneezing, and rhinorrhea) scores before and after the GCV treatment for the patients in the effective GCV treatment group. It can be seen that the nasal symptoms (rhinocleisis, rhinocnesmus, sneezing, and rhinorrhea) scores of the patients in the effective GCV treatment group are significantly improved after the GCV treatment. Results indicate that the GCV can not only ameliorate the clinical symptoms, but also allow routine anti-allergic medicines that are originally ineffective to re-play therapeutic effects.

### 2.3 the GCV has No Significant Effect on Eosinophils and ECP in the Nasal Secretions

In the common AR group, the percentage of eosinophils and ECP concentration in the nasal secretions decreased significantly after nasal spray hormone and oral antihistamine treatment. However, after the GCV treatment, the percentage of eosinophils and ECP in the GCV effective group and the GCV ineffective group have no significant change. The results of cytokines and eosinophils in nasal secretions of the three groups before and after treatment are shown in Table 2.

TABLE 2
Results of cytokines and eosinophils in nasal secretions of three groups before and after treatment are measured (χ ± SD)

| | Common AR group | | | | | |
|---|---|---|---|---|---|---|
| | Before | After | GCV effective group | | GCV ineffective group | |
| items | (treatment) | (treatment) | Before | After | Before | After |
| IL-2 (pg/mL) | 132.72 ± 31.33 | 148.22 ± 46.36 | 148.69 ± 43.59 | 132.85 ± 40.51 | 139.75 ± 59.26 | 125.13 ± 58.88 |
| IL-4 (pg/mL) | 182.36 ± 67.80^{†} | 135.63 ± 42.63^{†} | 109.67 ± 23.95 | 105.32 ± 25.44 | 151.81 ± 71.34 | 116.44 ± 53.74 |
| IL-5 (pg/mL) | 179.38 ± 222.95^{‡} | 28.04 ± 48.44^{‡} | 197.49 ± 388.67 | 134.24 ± 172.57 | 218.52 ± 189.78 | 70.26 ± 57.39 |
| IL-6 (pg/mL) | 22.10 ± 19.70 | 21.49 ± 33.27 | 39.10 ± 34.59 | 30.44 ± 27.04 | 49.62 ± 10.4 | 65.53 ± 44.86 |
| IL-13 (pg/mL) | 963.51 ± 524.36 | 738.07 ± 590.52 | 455.81 ± 190.16 | 397.80 ± 116.61 | 382.32 ± 104.87 | 282.34 ± 55.29 |
| IL-17A (pg/mL) | 81.22 ± 40.72 | 77.61 ± 40.03 | 55.38 ± 27.4 | 58.92 ± 43.93 | 59.88 ± 40.6 | 48.13 ± 18.73 |
| IL-28A (pg/mL) | 716.4 ± 274.34^{§} | 649.4 ± 251.47^{§} | 718.08 ± 263.12 | 740.68 ± 214.99 | 392.09 ± 218.69 | 447.41 ± 202.35 |
| IL-33 (pg/mL) | 617.71 ± 703.47 | 337.41 ± 207.46 | 277.39 ± 303.34 | 293.62 ± 396.68 | 583.28 ± 515.20 | 436.61 ± 500.63 |
| IFN-α (pg/mL) | 55.01 ± 46.25 | 78.80 ± 43.83 | 31.09 ± 28.65 | 32.77 ± 23.83 | 129.83 ± 170.32 | 57.51 ± 49.01 |
| IFN-β (pg/mL) | 45.79 ± 44.63^{¶} | 81.71 ± 70.87^{¶} | 26.49 ± 26.34 | 38.86 ± 54.29 | 19.58 ± 12.05 | 17.86 ± 8.57 |
| IFN-γ (pg/mL) | 81.76 ± 27.04 | 88.69 ± 35.05 | 181.83 ± 266.06 | 120.24 ± 116.93 | 106.77 ± 51.92 | 80.25 ± 44.49 |
| IFN-γ [symbol] (pg/mL) | 373.71 ± 272.15 | 503.56 ± 313.07 | 313.99 ± 194.7 | 331.23 ± 237.77 | 371.83 ± 248.73 | 363.86 ± 203.72 |
| Eotaxin (pg/mL) | 116.31 ± 75.34 | 100.5 ± 58.9 | 206.5 ± 212.85 | 153.51 ± 101.87 | 36.79 ± 25.41 | 50.35 ± 47.92 |
| ECP (ug/mL) | 42.23 ± 41.09* | 3.19 ± 4.98* | 42.38 ± 41.04 | 20.18 ± 18.42 | 82 ± 18.01 | 45.993 ± 37.12 |
| Nasal secretion | 75 ± 35.57** | 29.10 ± 43.79** | 90.54 ± 6.24 | 93.23 ± 11.99 | 56 ± 46.30 | 54 ± 44.54 |
| eosinophils (%) | | | | | | |

In table 2,
^{†}p = 0.01;
^{‡}p = 0.04;
^{§}p = 0.04;
^{¶}p = 0.02;
*p = 0.02;
**p = 0.01, p < 0.05 indicates that the cytokine detection is different and the difference is statistically significant.

### 2.4 GCV has No Significant Effects on Cytokines in Nasal Secretions

After treatment with the routine anti-allergic medicines, the concentrations of IL-4, IL-5 and IL-28A are decreased significantly and the concentration of IFN-β is increased (P<0.05) in nasal secretions of the common AR group, which is also consistent with the mechanism of Th2-related inflammation treatment. After the GCV treatment, all cytokines and inflammatory factors in nasal secretions of the GCV effective group and the GCV ineffective group do not change (Table 2), the eosinophils and the ECP do not decrease, IL-4, IL-5, the ECP and eosinophils are representative factors and cells of Th2 inflammation, and Th1, Th2 and Th17-related cytokines in nasal secretions do not change significantly. It suggests that the mechanism of the GCV in the intractable AR is different from that of the routine anti-allergic medicines, and also suggests that patients with the intractable AR may have other unknown mechanisms in addition to Th2-related inflammation. It is speculated that the unclear inflammatory mechanism may be related to the classical Th2-eosinophil mechanism and can greatly inhibit the therapeutic effect of the routine anti-allergic medicines. The GCV can effectively target the unclear inflammatory mechanism. When the inflammatory mechanism is broken by the GCV, the Th2-eosinophil mechanism returns to the track, and routine anti-allergic medicines are reactivated.

2.5 GCV May Regulate the Function of Nasal Mucosa in the Patients with the Intractable AR by Regulating Functions of Ciliated Cells or Leukocytes

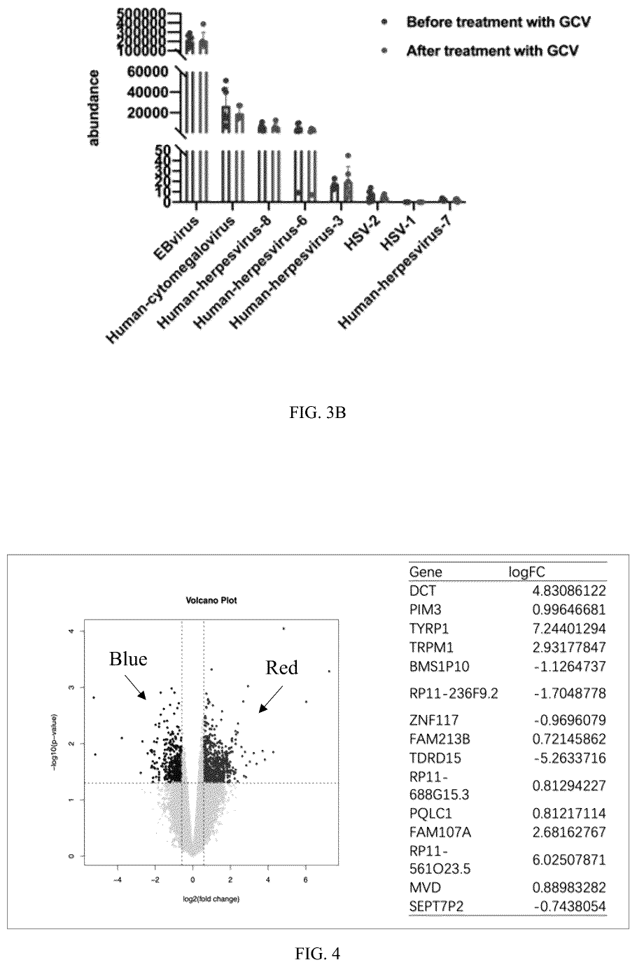

The human DNA virus sequences of nasal scraping cells in the common AR group and the GCV effective group before treatment and the human DNA virus sequences of nasal scraping cells in the GCV effective group before and after treatment are compared, which find no significant difference in the abundance of human DNA virus. The sequencing results are shown in FIGS. 3A-3B to FIG. 12. FIG. 3A is a comparison diagram of the abundance of genes of Epstein-Barr (EB) virus, human-cytomegalovirus, and human-herpes virus (HSV) 1, 2, 3, 6, 7, and 8 in the common AR group and the GCV effective group before treatment, and there is no significant difference in the enrichment of the genes of the EB virus, human-cytomegalovirus, and HSV 1, 2, 3, 6, 7, and 8 between the GCV effective group and the common AR group. FIG. 3B is a comparison diagram of the abundance of the genes of the EB virus, the human-cytomegalovirus, the HSV 1, 2, 3, 6, 7, 8 in the GCV effective group before and after treatment, and there is no statistical difference in the enrichment of the genes of the EB virus, the human-cytomegalovirus, the HSV 1, 2, 3, 6, 7, 8 before and after the GCV treatment.

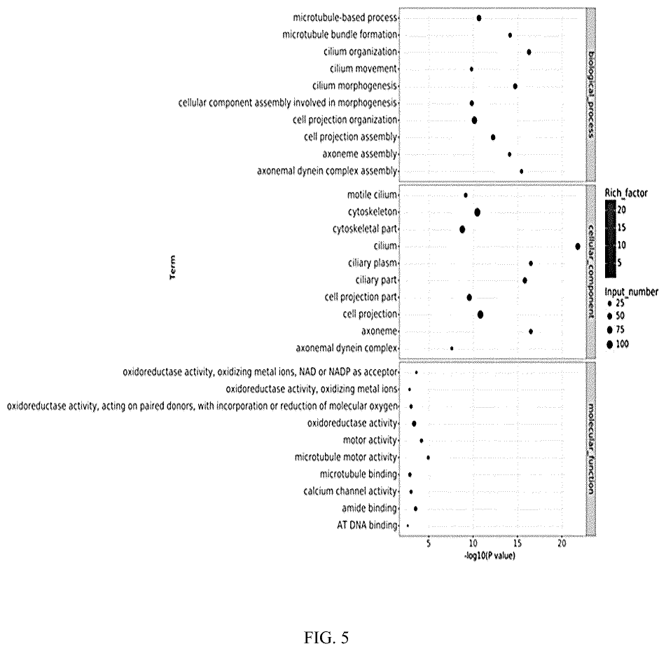

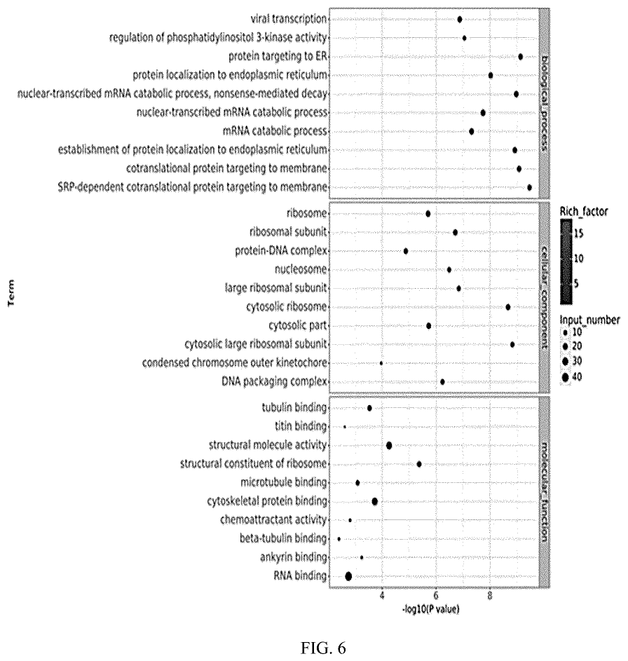

The ribonucleic acid sequencing (RNA-seq) of nasal scraping cells in the common AR group, the GCV effective group and the GCV ineffective group before treatment is compared. The left side of FIG. 4 is the volcano plot drawn by the differential genes before treatment in the GCV effective group (n=6) and the common AR group (n=5). The red indicates that the gene is highly expressed in the GCV effective group, and the blue indicates that the gene is lowly expressed in the GCV effective group. The first 15 differential genes are shown on the right side of FIG. 4. FIG. 5 shows a pathway of up-regulated differential gene enrichment in the GCV effective group compared with the common AR group. FIG. 6 shows a pathway of down-regulated differential gene enrichment in the GCV effective group compared with the common AR group. It is found that the differential genes between the GCV effective group and the common AR group are DCT, TYRP1, RP11-561023.5, FAM107A, etc., and the differential genes are mainly concentrated in the biological function process related to cilia.

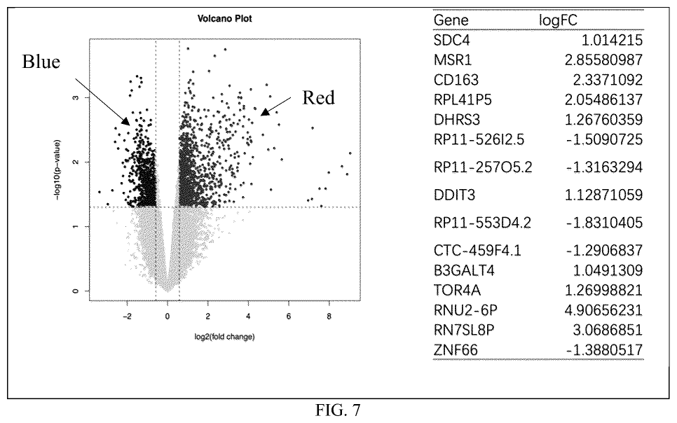

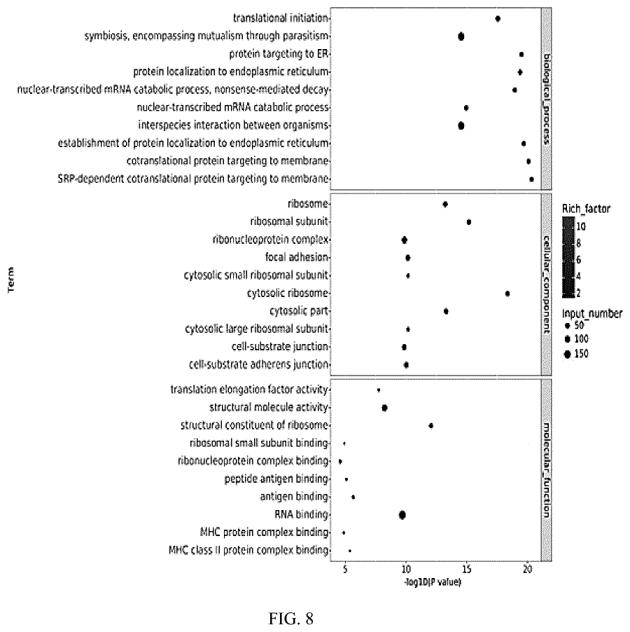

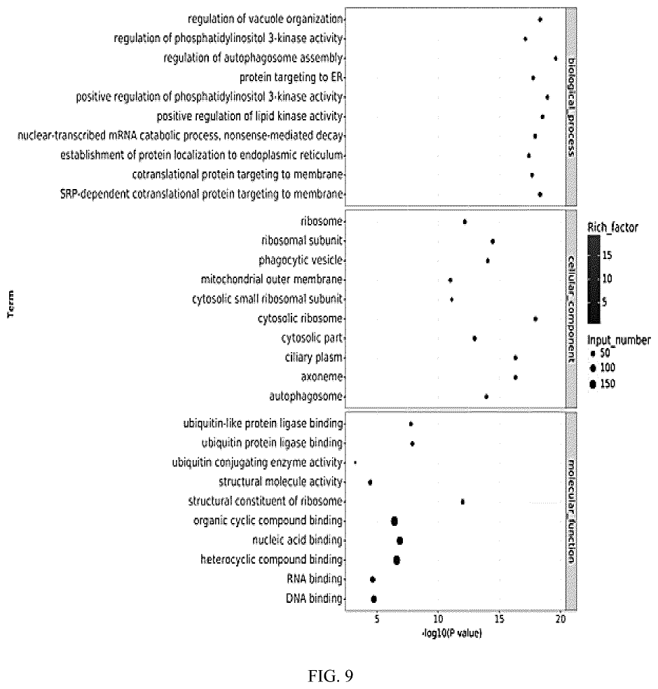

FIG. 7 left is a volcanic plot drawn by RNA-seq differential genes of the nasal scraping cells before the treatment in the GCV effective group (n=6) and the GCV ineffective group (n=3). The red indicates that genes are highly expressed in the GCV effective group, and the blue indicates that genes are lowly expressed in the GCV effective group. The first 15 differential genes are shown on the right of FIG. 7. FIG. 8 shows a pathway of up-regulated differential gene enrichment in the GCV effective group compared with the GCV ineffective group. FIG. 9 shows a pathway of down-regulated differential gene enrichment in the GCV effective group compared with the GCV ineffective group. It can be seen that the differential genes between the GCV effective group and the GCV ineffective group are MSRI, CD163, RNU2-6P, RPL41P5, etc. The differential genes are mainly concentrated in biological processes related to protein regulation. The expression of CD163 is up-regulated in the GCV effective group. CD163 is a specific marker of monocytes/macrophages, mainly expressed in cells involved in the inflammatory response, which means that monocytes/macrophages may be involved in the mechanism of the intractable AR and may also be associated with the GCV treatment.

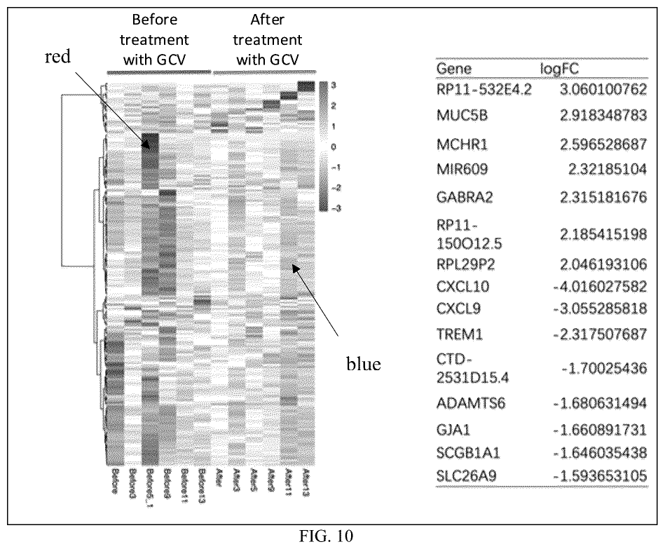

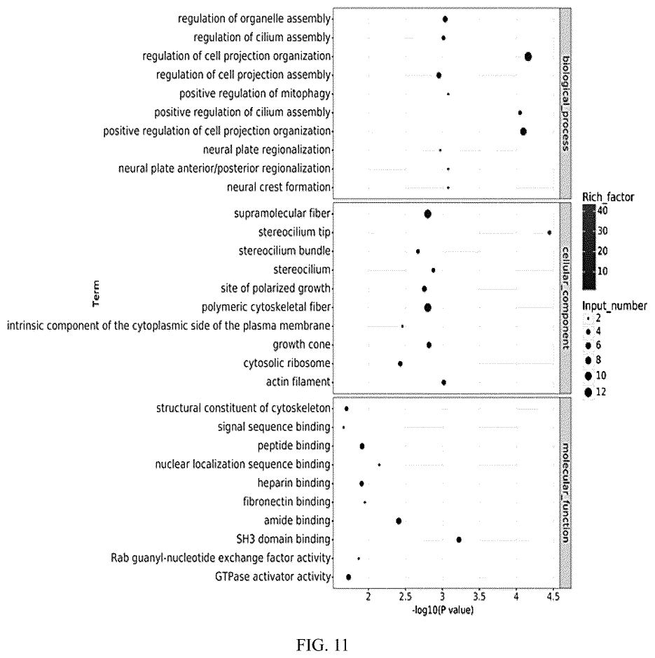

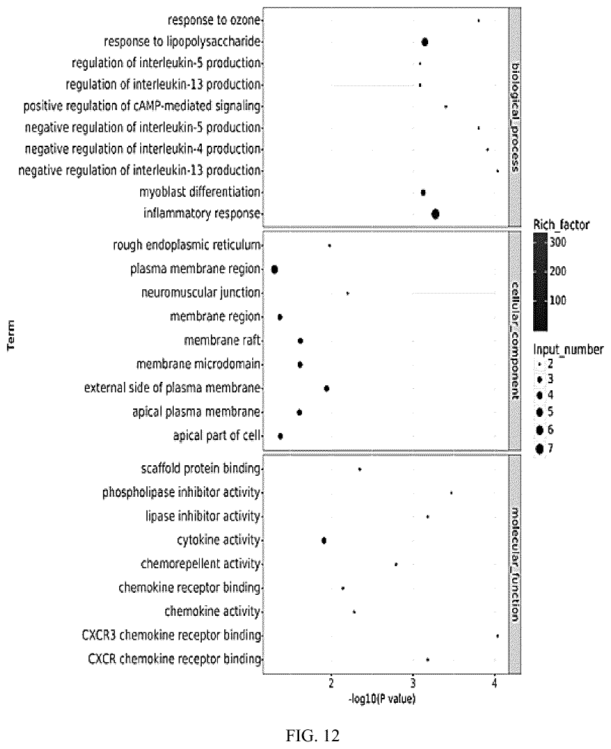

FIG. 10 shows a cluster heat map of RNA-seq of differential genes of the nasal scraping cells before and after treatment in the GCV effective group (n=6). The red indicates gene overexpression and the blue indicates low gene expression. The top 15 differentially expressed genes in the GCV effective group before and after treatment are shown on the right of FIG. 10. FIG. 11 shows a pathway of up-regulated differential gene enrichment after GCV treatment compared with that before GCV treatment. FIG. 12 shows a pathway of down-regulated differential gene enrichment after GCV treatment compared with that before GCV treatment. It can be seen that after GCV treatment, 160 genes are down-regulated and 25 genes are up-regulated. The down-regulated differential genes are mainly cilia-related cytological functions; the up-regulated differential genes are mainly enriched in biological functions such as negative regulation of IL-5 and IL-13 production, response to ozone, and molecular function of chemokine binding. Differential genes are significantly enriched in phagosomes, Toll-like receptors and leukocytes transendothelial migration pathways. The top 15 differential genes are listed as follows: RP11-532E4.2, MUC5B, MCHR1, MIR609, GABRA2, RP11-150012.5, RPL29P2, CXCL10, CXCL9, TREM1, CTD-2531D15.4, ADAMTS6, GJA1, SCGB1A1, and SLC26A9. It can be seen from the results that MUC5B related to goblet cells is down-regulated after the GCV treatment. However, CXCL9 and CXCL10 are up-regulated after the GCV treatment (FIG. 10). Therefore, it is speculated that the changes of the above pathways/chemokines may be related to the mechanism of the GCV treatment.

2.6 GCV can Regulate the Immune Status of the Patients with the Intractable AR by Increasing Regulatory T Cells (Tregs, Also Referred to Treg Cells) in Peripheral Blood

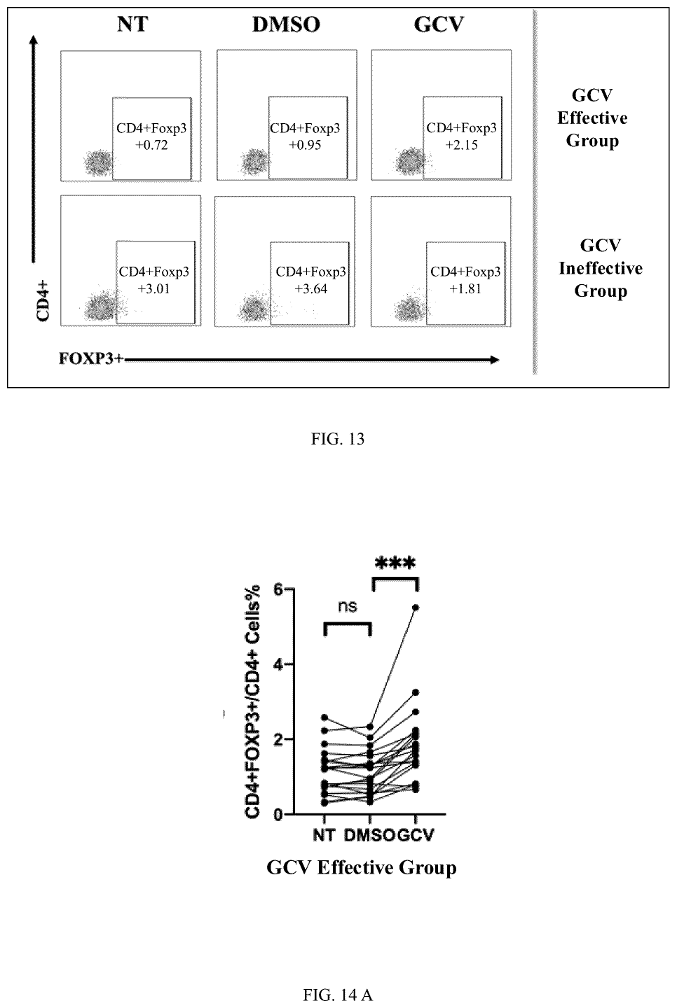

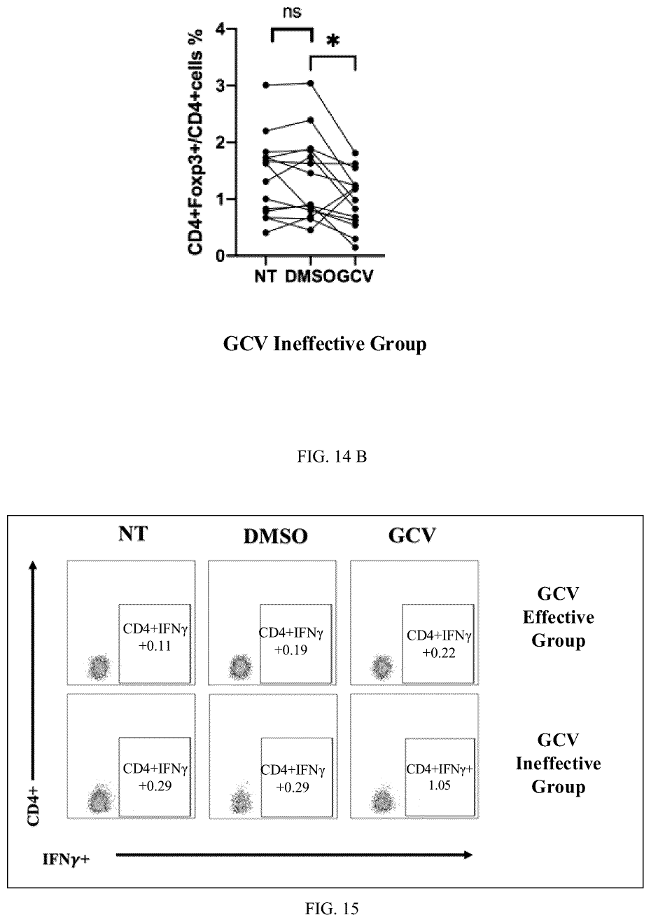

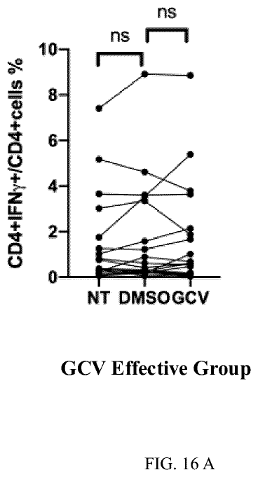

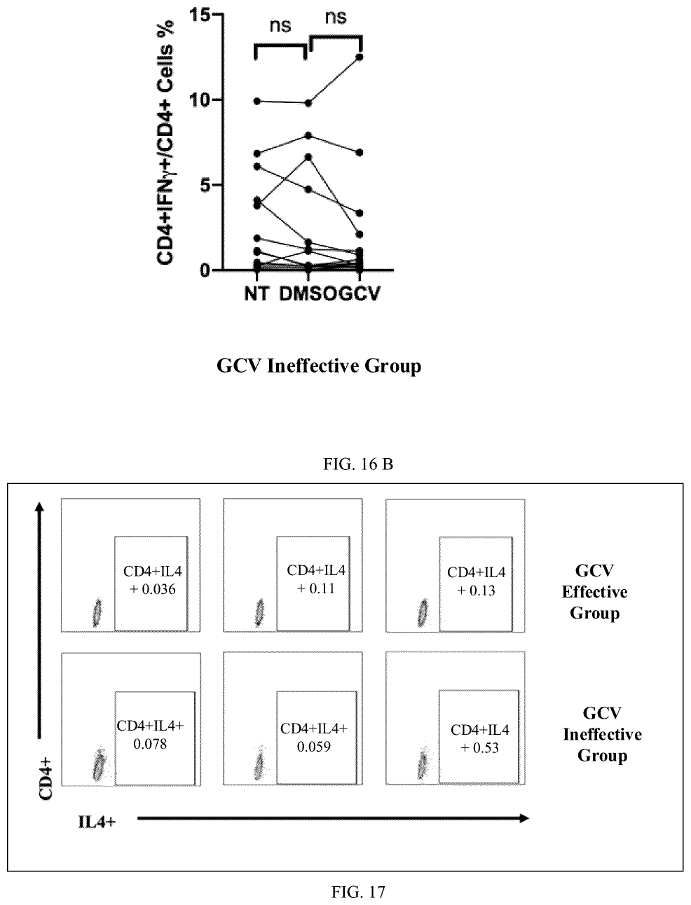

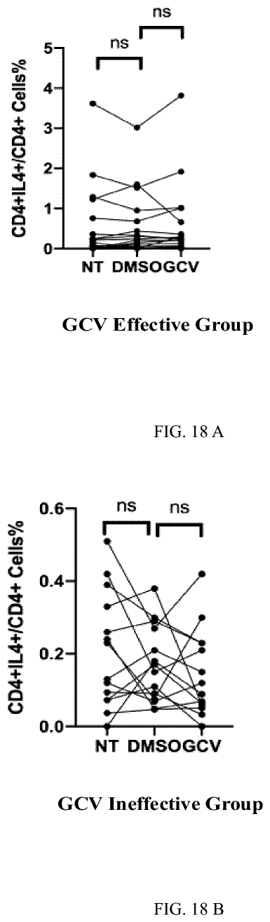

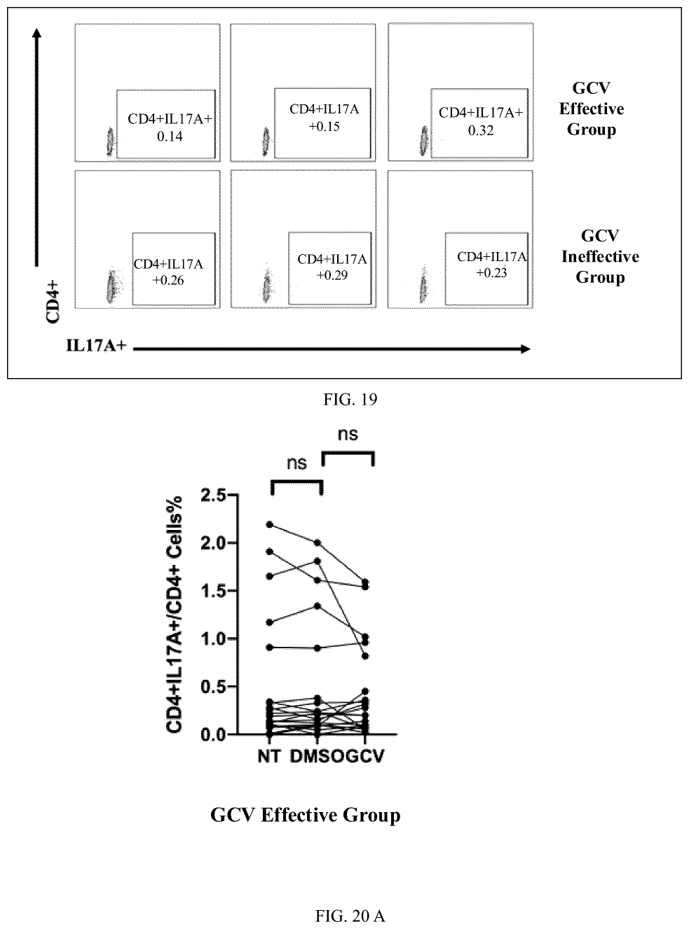

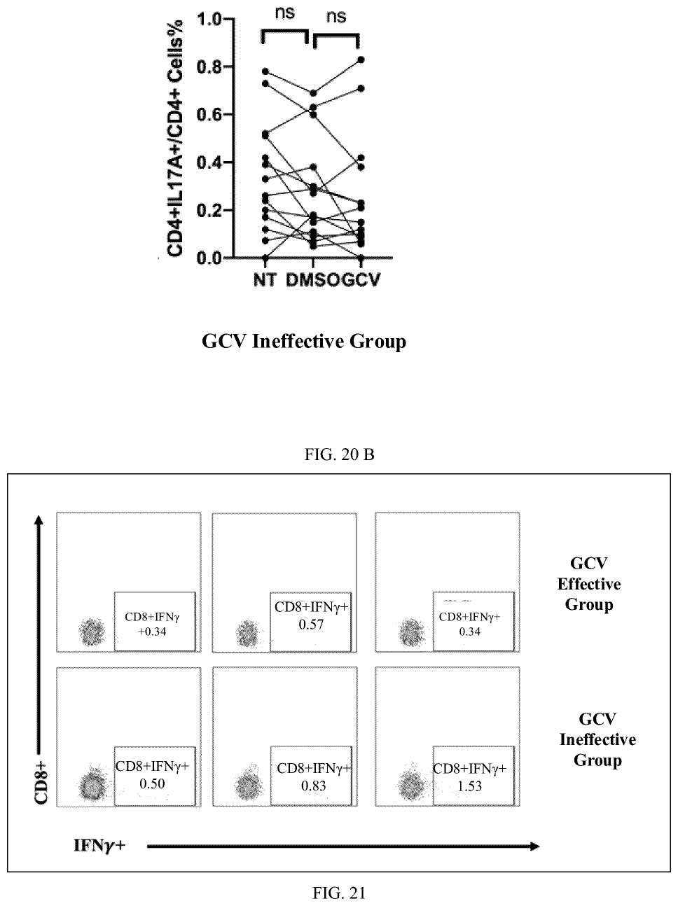

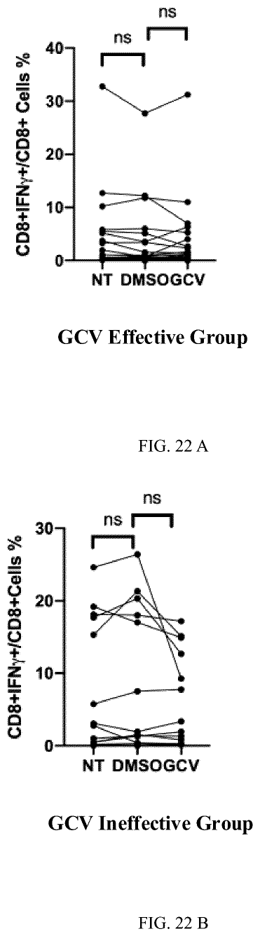

There is no difference between the NT well and the DMSO well in the in vitro medicine test of the PBMCs. FIGS. 13-22 show the comparison of the results of GCV stimulation of the PBMCs in vitro between the GCV effective group and the GCV ineffective group. It can be seen that the percentage of cluster of differentiation 4⁺ (CD4⁺) Forkhead box protein P3⁺ (FOXP3⁺) cells/CD4⁺ cells of the GCV well in the GCV effective group PBMCs (n=26) is significantly higher than that in the DMSO well (p<0.001). In the GCV ineffective group PBMCs (n=14), the percentage of CD4⁺FOXP3⁺ cells/CD4⁺ cells in the GCV well is significantly lower than that in the DMSO well (p<0.05). The correlation between the proportion of Foxp3⁺ Treg cells and the improvement of clinical symptoms is analyzed, which finds that the increase in the percentage of Foxp3⁺ Tregs has a medium-high correlation with ΔTNSS and ΔVAS (ΔTNSS r=0.6085, ΔVAS r=0.7050, P<0.001). The above results indicate that Tregs may be involved in the inflammatory mechanism of the intractable AR, and the GCV may play its therapeutic role by enhancing the activation of Treg cells.

FIGS. 13-22 show that there is no significant change in the percentage of CD4⁺IFNγ⁺ cells, CD4⁺-IL4⁺ cells, CD4⁺IL17A⁺ cells, CD8⁺IFNγ⁺ cells/CD4⁺ in the in vitro medicine test of the GCV effective group and the GCV ineffective group in clinical.

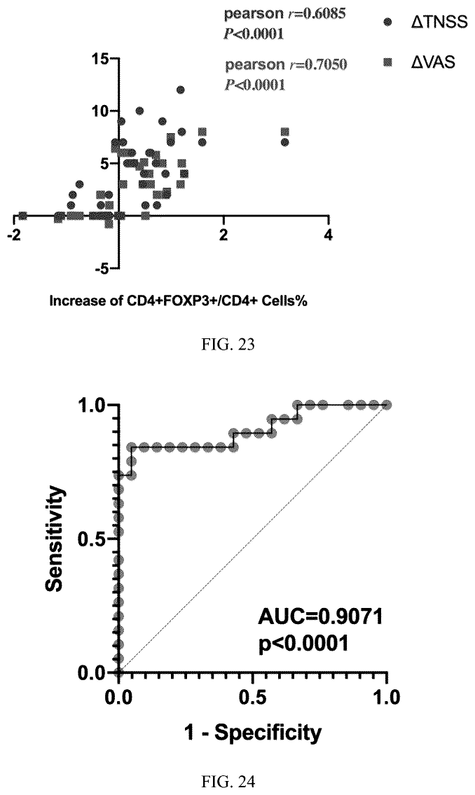

FIG. 23 illustrates a scatter diagram of changes in TNSS and VAS before and after treatment with GCV and changes in proportion of Foxp3⁺ Treg cells stimulated by the GCV. It can be seen that the proportion of Foxp3⁺ Treg cells stimulated by the GCV is significantly correlated with the improvement of the clinical symptoms (ΔTNSS r=0.6085, ΔVAS r=0.7050, P<0.001).

The receiver operating curve characteristic (ROC) curve is drawn by changes in the proportion of Foxp3⁺ Treg cells in the GCV effective group and the GCV ineffective group to evaluate the value of the change in the proportion of Foxp3⁺ Treg cells in predicting the efficacy of the GCV treatment. As shown in FIG. 24, the area under the curve of GCV efficacy is 0.9071 (P<0.0001) with a 95% confidence interval of 0.8077 to 1.000. It is found that the value of 0.045 for the proportional increase of Foxp3 Treg cells in PBMCs stimulated by the GCV in vitro is an optimal threshold to distinguish between effective and ineffective GCV treatments, the sensitivity is 84.2%, and the specificity is 95.2%. The change trend of CD4⁺ FOXP3⁺ Treg cells after GCV stimulation of PBMCs in vitro may be a good objective predictor of the clinical efficacy of GCV in the treatment of the intractable AR.

The GCV has brought great convenience to the treatment of the intractable AR in clinical practice. Its price is low, and in the long-term clinical use, the safety of medication has been fully confirmed. According to clinical observations, about 65% of the intractable AR patients respond well to the GCV treatment. In addition, the GCV medicine test of the PBMCs in vitro can more accurately screen out the potential effective population. Moreover, it is found that most patients can maintain long-term therapeutic effect after receiving GCV treatment. In summary, the study demonstrates the therapeutic effect of GCV on intractable AR and confirms that the GCV can indeed be used as a therapeutic medicine for the intractable AR.

### Specific Cases are as Follows.

### Case 1

Zeng **, male, 13 years old, was admitted to the department of otolaryngology with a 1.5-year history of rhinitis symptoms. The patient had rhinocleisis, rhinocnesmus, sneezing, and rhinorrhea many times a day, lasting for several hours each time. The patient complained that the use of nasal spray hormones and oral antihistamines in the early stage of the disease can effectively control the nasal symptoms, and then the duration of medication was gradually shortened. Later, other types of the nasal spray hormones and the oral antihistamines were used, and leukotriene receptor antagonists were added, and even Chinese herbal medicine and sublingual immunotherapy (SLIT) were used, which can not effectively ameliorate the nasal symptoms. The patient's parents and the patient were very painful for the situation, and they had not been able to find a good treatment for a long time. A skin prick test (df. +++; dp. +++) is performed at the outpatient visit, a total serum IgE detection was 255 kilounit per liter (kU/L), nasal secretion smear showed eosinophils/inflammatory cells>90%, VAS was 8 cm, ARCT was 5 points, TNSS was 8 points (rhinocleisis 3 points, rhinocnesmus 1 point, sneezing 1 point, nasal secretion 3 points). Since Oct. 7, 2021, the patient receives the GCV treatment for 14 days (250 mg/time, twice a day), and the patient's nasal symptoms were significantly ameliorated. TNSS decreased to 3 points (rhinocleisis 1 point, rhinocnesmus 1 point, sneezing 0 point, rhinorrhea 1 point); VAS decreased to 4.5 cm and ARCT was 21 points. The patient felt that the nasal symptoms were ameliorated significantly. After that, the patient was treated with the routine anti-allergic medicines for 14 days, the patient felt that the nasal symptoms were further ameliorated, TNSS decreased to 2 points (rhinocleisis 1 point, rhinocnesmus 0 point, sneezing 0 point, rhinorrhea 1 point), VAS decreased to 2.5 cm, and ARCT was 24 points. After a period of discontinuation of the medication, no worsening of symptoms was observed. At the follow-up visit on Jan. 25, 2023, the patient's father told us that the child now had occasional AR, and the onset time was short. Anti-allergic medicines could quickly control the symptoms, and the effect of medication was restored to the initial onset of the AR.

### Case 2

Wei **, female, 37 years old, was admitted to the department of otolaryngology for rhinocnesmus, rhinocleisis and paroxysmal sneezing for 5 years. The patient complained previous use of nasal spray hormones and oral antihistamines, which were continued for 3-5 days at a time, and the nasal symptoms were slightly alleviated. However, the effect of the medicine gradually decreased, and the symptoms were almost not ameliorates after 2 weeks of continuous medication. She also tried to replace other brands of nasal spray hormones, oral antihistamines, and added leukotriene receptor antagonists, but the nasal symptoms were not significantly ameliorated. The patient was extremely sensitive to cold air, and it was easy to stimulate nasal allergic symptoms in air-conditioned rooms. A skin prick test (d.p +++; cockroach+) is performed at the outpatient visit, a total serum IgE detection was 111.96 kU/L, nasal secretion smear showed eosinophils/inflammatory cells>95%, VAS=7 cm, ARCT was 7 points, TNSS was 8 points (rhinocleisis, rhinocnesmus, sneezing, rhinorrhea each 2 points). Since May 31, 2022, the patient begins to receive the GCV treatment for 14 days (500 mg/time, twice a day), and the patient's nasal symptoms ameliorated significantly. TNSS decreased to 4 points (rhinocleisis 2 points, rhinocnesmus 2 points, sneezing 0 points, and rhinorrhea 0 points), while VAS decreased to 4 cm and ARCT was 20 points. On Jun. 14, 2022, the patient continued to use anti-allergic medicines (nasal spray hormone plus oral antihistamines) for 14 days. After 14 days of treatment, the patient's nasal symptoms continued to ameliorate. The TNSS decreased to 1 point (rhinocleisis 1 point, rhinocnesmus 0 point, sneezing 0 point, rhinorrhea 0 point), VAS decreased to 2 cm, ARCT was 25 points. After a period of discontinuation of the medication, the symptoms did not recur. At a return visit on Jan. 30, 2023, the patient complained of occasional allergic symptoms, and the use of anti-allergic medicines at the time of onset was easily controlled. The patient felt that conscious nasal symptoms had been greatly ameliorated and had less sensitive to cold air, and can rest in the air-conditioned room.

### Case 3

Deng **, an 8-year-old male child, had visited the department of otolaryngology with rhinocleisis, rhinocnesmus, sneezing and rhinorrhea for more than 5 years. His mother complained that since the age of 2-3 years old, he could not sleep peacefully at night due to rhinocleisis and rhinocnesmus, and often turns over frequently due to rhinocleisis after sleep, which seriously affected the quality of life of the child. Various types of nasal spray hormones, oral antihistamines and anti-leukotriene medicines had been used, but the symptoms had not ameliorated after treatment. During the period, the child also underwent adenoidectomy and tonsillectomy, but the symptoms did not ameliorate after the operation. Outpatient skin prick test showed dust mites +++ and house dust mite +++, food intolerance test showed eggs (++), milk (+), wheat (+), soybean (+), cod (+). Nasal secretion smear showed eosinophils/inflammatory cells>90%, TNSS was 12 points (rhinocleisis 3 points, rhinocnesmus 3 points, sneezing 3 points, rhinorrhea 3 points), VAS was 10 cm, ARCT was 5 points. From Jul. 26, 2022, after 14 days of GCV treatment (250 mg/time, twice a day), the nasal symptoms of the child were ameliorated, the TNSS decreased to 9 points (rhinocleisis 2 points, rhinocnesmus 1 point, sneezing 3 points, rhinorrhea 3 points), VAS decreased to 5 cm, ARCT was 22 points. The patient complained of reduced rhinocleisis and rhinocnesmus at night, and the quality of sleep was also greatly improved. However, sneezing and rhinorrhea did not change significantly. Continued to give routine anti-allergic medicines for 14 days. The nasal symptoms of the children were significantly ameliorated, and the TNSS decreased to 3 points (rhinocleisis 1 point, rhinocnesmus 0 point, sneezing 1 point, rhinorrhea 1 point). In this situation, the VAS decreased to 2.5 cm, and the ARCT was 25 points. The patient felt that the nasal symptoms were significantly ameliorated, and the quality of sleep at night was significantly improved. At a telephone follow-up visit on Feb. 1, 2023, the mother of the child was informed that the child no longer had obvious nasal allergic symptoms and that she slept peacefully at night.

### Case 4

Qian **, female, 36 years old, was admitted to the department of otolaryngology due to persistent bilateral rhinocleisis and water-like nasal secretions for 2 years. The patient complained of rhinocnesmus and paroxysmal sneezing when exposed to cold air, and the symptoms continued for about 2 hours after leaving the cold air environment. Persistent symptoms of the bilateral rhinocleisis and water-like nasal secretions seriously interfered with daily life. Since the disease, nasal spray hormones and oral antihistamines had been regularly used, but the symptoms had not ameliorated. She switched to other brands of the nasal spray hormones and antihistamines, and even injected Omalizumab, and her nasal symptoms still did not ameliorate. Her rhinocleisis and rhinorrhea had seriously affected her work and life. The outpatient test results showed that the total serum IgE was 92.61 international units per liter (IU/L), and the nasal secretion smear showed eosinophils/inflammatory cells>90%. TNSS was 7 points (rhinocleisis 3 points, rhinocnesmus 1 point, sneezing 1 point, rhinorrhea 2 points), and VAS was 9 cm. From Aug. 12, 2022, after 14 days of the GCV treatment (500 mg/time, twice a day), the patient's nasal symptoms were significantly ameliorated, TNSS decreased to 2 points (rhinocleisis 1 point, rhinocnesmus 0 point, sneezing 0 point, rhinorrhea 1 point), VAS decreased to 1.5 cm, ARCT was 24 points. The patient complained of alternating rhinocleisis in the morning and evening, occasional rhinorrhea, sneezing when exposes to cold air, no rhinocnesmus, and the symptoms disappeared immediately after leaving the cold air environment. The patient continued to be treated with anti-allergic medicines. After 7 days of the nasal spray hormone, the nasal symptoms disappear completely, and then the medicine was discontinued. At the follow-up on Jan. 31, 2023, the patient complained of no obvious nasal discomfort for a long time after medicine withdrawal, and occasionally had rhinocleisis, but it could be alleviated by taking the anti-allergic medicines.

### Case 5

Liu **, male, 39 years old, had 10-year history of rhinocnesmus and rhinorrhea, rhinocleisis and rhinorrhea had been aggravated for 3 years. The patient complained of severe rhinocleisis and rhinorrhea every autumn. Nasal symptoms were mild in other seasons. During the onset of rhinitis, nasal symptoms could not be ameliorated even if nasal spray hormones and oral antihistamines were regularly used. The patient had tried to replace and use a variety of therapeutic medicines, but none of them had been effective. He was treated with Omalizumab without any improvement in nasal symptoms. Serological allergen detection showed that Humulus pollen graded 4, crab graded 2, a total serum IgE was 161.66 IU/mL, nasal secretion smear showed eosinophil/inflammatory cells>90%. TNSS was 7 points (rhinocleisis 3 points, rhinocnesmus 0 points, sneezing 3 points, rhinorrhea 1 point), VAS was 10 cm, and ARCT was 5 points. From Sep. 9, 2022, after 14 days of the GCV treatment (500 mg/time, twice a day), the patient felt a significant improvement in nasal symptoms. The TNSS decreased to 2 points (rhinocleisis 1 point, rhinocnesmus 0 point, sneezing 0 point, rhinorrhea 1 point), while the VAS decreased to 2.2 cm, and the ARCT was 24 points. The patient complained that the symptoms of the previous autumn rhinitis could last until late October, and all kinds of medicine treatments were ineffective, so the treatment plan was satisfactory. At the follow-up on Jan. 31, 2023, the patient was informed that there was only a slight rhinocleisis and a small amount of clear rhinorrhea in the morning. The duration was very short, and the symptoms could disappear without medication. Because the patient is not currently in the high incidence season, follow-up observation will continue.

What is claimed is:

1. An application method of ganciclovir, comprising:
preparing a medicine by using the ganciclovir to treat allergic rhinitis.

2. The application method as claimed in claim 1, wherein clinical symptoms of the allergic rhinitis comprise at least one selected from a group consisting of rhinocleisis, rhinocnesmus, sneezing, and rhinorrhea.

3. The application method as claimed in claim 1, comprising:
one of using the ganciclovir as an only active component to treat the allergic rhinitis and using the ganciclovir in combination with another medicine ameliorating the allergic rhinitis to treat the allergic rhinitis.

4. The application method as claimed in claim 3, wherein the using the ganciclovir in combination with the another medicine ameliorating the allergic rhinitis to treat the allergic rhinitis comprises:
one of using the ganciclovir first, then using the another medicine ameliorating the allergic rhinitis as needed and using the ganciclovir in combination with the another medicine ameliorating the allergic rhinitis simultaneously.

5. The application method as claimed in claim 4, wherein the another medicine ameliorating the allergic rhinitis comprises at least one of a nasal spray hormone and an oral antihistamine.

6. The application method as claimed in claim 5, wherein the nasal spray hormone comprises one of mometasone furoate, budesonide, triamcinolone acetonide, and fluticasone; and the oral antihistamine comprises one of loratadine, cetirizine, and ebastine.

7. The application method as claimed in claim 4, wherein the using the ganciclovir first, then using the another medicine ameliorating the allergic rhinitis as needed comprises:
using the ganciclovir for 7-14 days first, then continually using the another medicine ameliorating the allergic rhinitis for 7-14 days as needed.

8. The application method as claimed in claim 4, wherein forms of the prepared medicine using the ganciclovir comprise:
capsules, granules, tablets, pulvis, ointments, powders, and aqueous solutions prepared by mixing pharmaceutically acceptable excipients with one of the ganciclovir as the only active component and a combination of the ganciclovir and other oral medicines ameliorating the allergic rhinitis.

9. The application method as claimed in claim 1, wherein a dosage of the ganciclovir is in a range of 250-500 milligrams per time, twice a day for 14 years old and above by oral administration, and 125-250 mg per time, twice a day for under 14 years old by oral administration.
